(19) United States
(12) Patent Application Publication
(10) Pub. No.: US 2017/0014340 A1
(43) Pub. Date: Jan. 19, 2017

(54) Orally Disintegrating Tablet of Nabilone Comprising Mannitol-Based Granules

(71) Applicant: Pharmascience Inc., Montréal, Québec (CA)

(72) Inventors: Ousmane Diallo, Montréal, Québec (CA); Mathew Philip, Montréal, Québec (CA); Naresh Talwar, Kirkland, Québec (CA)

(73) Assignee: Pharmascience Inc., Montréal, Québec (CA)

(21) Appl. No.: 15/123,984

(22) PCT Filed: Mar. 4, 2015
(86) PCT No.: PCT/CA2015/000134

§ 371 Date: Sep. 6, 2016

(30) Foreign Application Priority Data
Mar. 4, 2014 (CA) 2,845,443

(51) Int. Cl. A61K 9/00; A61K 9/20; A61K 31/352

(52) U.S. Cl. CPC A61K 9/0056; A61K 31/352; A61K 9/2018; A61K 9/2027; A61K 9/2095

(57) ABSTRACT

Orally disintegrating medicaments comprising Nabilone allow for improved treatment of nausea arising from chemo therapy for cancer. The medicaments comprise appropriate excipients such that the medicament disintegrates in the mouth in 30 seconds or less, while exhibiting sufficient stability for storage. In a preferred embodiment, the medicament is in the form of a tablet formed from granules. The granules consist of an intra-granular fraction comprising nabilone, mannitol, and polyvinyl pyrrolidone and an extra-granular fraction comprising mannitol, calcium silicate, crospovidone, and magnesium stearate. Processes for manufacturfing such medicaments are also disclosed.

### FIELD OF THE INVENTION

The present invention relates to pharmaceutical compositions comprising Nabilone, and specifically to the orally disintegrating tablet formulation comprising nabilone, or a pharmaceutically acceptable salt thereof, which dissolves or disintegrates in oral cavity less than 30 seconds.

The present invention also relates to a manufacturing process for the preparation of an orally disintegrating pharmaceutical composition comprising nabilone along with at least one pharmaceutically acceptable excipient. The orally disintegrating tablet of nabilone is for use in the therapeutic treatment of chemotherapy-induced nausea and vomiting as to increase of the life quality of patients who have difficulties to swallow gelatin capsules.

### BACKGROUND OF THE INVENTION

Nabilone is an orally active synthetic cannabinoid that have complex effect in the central nervous system, which is indicated for therapeutic use as an antiemetic and anti-anxiety agent and as an adjunct analgesic for neuropathic pain.

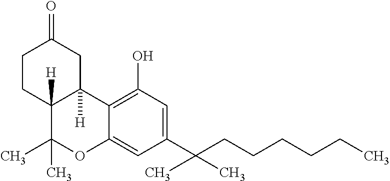

Nabilone was approved in 1985 by the U.S. Food and Drug Administration (FDA) for treatment of chemotherapy-induced nausea and vomiting that has not responded to conventional antiemetics. Also, it is approved for use in treatment of anorexia and weight loss in patients with AIDS. The positive effect of using nabilone for the treatment of chemotherapy-induced nausea and vomiting (CINV) and increase of the life quality of patients was shown in several clinical studies. In Canada, United States, United Kingdom and Mexico, nabilone is marketed under the trade name Cesamet® in the form of gelatin capsules.

Nabilone is not well absorbed through the intestine upon oral administration. Takker et al., J. Pharma. Pharmac. 29, 78 (1977) describe useful formulations of nabilone with a dispersion in polyvinylpyrrolidone. U.S. Pat. No. 4,195,078, discloses a method of formulating nabilone for oral administration comprises dissolving nabilone and polyvinylpyrrolidone or polyethylene glycol in anhydrous ethanol and using the thus-formed viscous solution to granulate a pharmaceutically-acceptable ethanol-insoluble excipient by thoroughly mixing the solution with the excipient, and then drying the thus-formed granulation.

Canadian Patent No. 1124178, discloses a granulation process using a solid dispersion of one part of nabilone in 2:20 parts of PVP. The granulation solution was used to wet granulate other pharmaceutical excipients. The process described in that patent involve drying of wet granules, sizing and final blending steps to make a suitable blend for filing in gelatin capsules.

The aqueous solubility of nabilone is extremely low, less than 0.5 pg/ml at 25° C. The occurrence of at least four distinct polymorphic forms with different bioavailability characteristics further complicates the development of a stable dosage form. Until present, due to its poor solubility in water, nabilone is available only as gelatin capsule which is highly disadvantageous especially for patients suffering from nausea who have difficulties to swallow these capsules.

The oral disintegrating tablets (ODT) serves as an alternative dosage form for patients who experience Dysphagia (difficulty in swallowing) or for where compliance is a known issue and therefore an easier dosage form to take ensures that medication is taken. Common among all age groups, dysphagia is observed in about 35% of the general population, as well as up to 60% of the elderly institutionalized population and 18-22% of all patients in long-term care facilities. An additional reason to use an ODT's is the convenience of a tablet that can be taken without water (R. Thakur, J. Applied Pharmaceutical Science, Volume 1, Issue 1, March 2011).

In the development of an oral disintegrated dosage form, the choice of the core excipients is extremely important. Several aspects of the finished dosage form must be considered such as the nature of the active pharmaceutical ingredient (API), the intended delivery method of the API (immediate release), and the manufacturing process. Highly water soluble diluents can improve the mouth feel. Tablet compressed at lower hardness may have high friability, on the other hand, high hardness may prolong disintegration time. In generic nabilone capsules the filler is pregelatinized starch, that excipient is known to extend the disintegration time because of his gelling ability. Also, the ratio nabilone/PVP K30 in the solid dispersion is fixed because of that ratio was shown to give an amorphous form of nabilone and improved solubility. Povidone, also have an impact on disintegration time because of its inherent binding characteristics

Orally Disintegrating Tablets (ODT) allows to improve patient compliance, in particular with pediatric, geriatric, and institutionalized patients. Patients undergoing chemotherapy are taking usually several drugs at the same time and therefore may have treatment compliance problems. Also, after chemotherapy, wide type of cancer patients often have swallowing and chewing difficulties. Swallowing impairments can compromise treatment compliance and lead to poor clinical outcome. Orally Disintegrating Tablet (ODT) dosage forms can therefore be suitable for those patients to better follow treatments. Overcoming dysphagia problems for patients undergoing chemotherapy is a key for good clinical outcome. For patients with chemotherapy-induced nausea, there is no orally disintegrated tablet dosage form of nabilone in the current market. In order to maximize patient compliance, it is a need for a formulation of nabilone orally disintegrating tablet that is stable and bioequivalent to nabilone capsules, with disintegration time less than 60 seconds, with good mouth feel and friability that did not exceed 1%.

One aspect of the present invention is to provide nabilone dosage form in an orally disintegrating tablet formulation as an alternative to commercially available cannabinoid-containing oral dosage forms as to overcome problems for patients with chemotherapy-induced nausea who have difficulties to swallow these capsules.

The present invention provides a process of manufacturing an orally disintegrating Nabilone dosage form, which is simple and less expensive method. Also provides a stable orally disintegrating tablet of nabilone which is bioequivalent to Cesamet® (1 mg) capsules, is convenient to take, is quick in absorption and takes effect quickly.

### SUMMARY OF THE INVENTION

One aspect of the present invention is to provide the nabilone orally disintegrating pharmaceutical composition, which is fast disintegrating and absorbed and providing greatly convenience for patients, as to avoid dysphagia problems for patients with chemotherapy-induced nausea.

Another aspect of the present invention is to provide a nabilone orally disintegrating pharmaceutical composition that disintegrates less than 30 seconds in an oral cavity and that is bioequivalent to Cesamet®.

A further aspect of the present invention provides a method of manufacturing an orally disintegrating nabilone tablet pharmaceutical composition.

An aspect of the present invention provides an orally disintegrating pharmaceutical composition comprising a nabilone or a pharmaceutically acceptable salt thereof, at least one disintegrant, at least one filler, at least one binder and a lubricant, wherein the tablet is orally administrated through disintegration in the mouth by saliva or water in a similar amount to the saliva.

A further aspect of the present invention provides an orally disintegrating tablet comprising:

(i) an intra-granular fraction, wherein said fraction comprising:
a) nabilone or a pharmaceutically acceptable salt thereof; and
b) at least one filer,
c) at least one binder, and

(ii) an extra-granular fraction, wherein said fraction comprising:
a) at least one filer;
b) at least one disintegrant, and
c) at least one other pharmaceutically acceptable excipient,

wherein the tablet disintegrated fast and takes effect quickly by reducing patient discomfort in dysphagia problems for patients with chemotherapy-induced nausea. Embodiments of the present invention may increase compliance in patients exhibiting dysphagia.

Preferably, the active pharmaceutical ingredient is nabilone or a pharmaceutically acceptable salt thereof and is present in an amount ranging from 0.01 mg to 10.0 mg. Preferably, nabilone is present in an amount ranges from 0.1 mg to 5.0 mg, more preferably from 0.25 mg to 1.0 mg.

In another aspect of the present invention, the ODT formulation comprises from 0.1% to 0.5% w/w of nabilone, from 60.0% to 80.0% w/w of mannitol, from 1.0% to 10.0% w/w of povidone, from 5.0% to 10.0% w/w calcium silicate, from 10.0% -20.0% w/w of crospovidone XL, and from 0.5% to 2.0% w/w/ of magnesium stearate.

In another aspect of the present invention the orally disintegrating nabilone tablet has an in vitro dissolution profile, that provides more than 95% of the active ingredient released within 10 minutes, using USP apparatus Type II, placing the tablet in 1000 ml of 0.1% tween 80 at 37° C.

In a further aspect of the present invention the orally disintegrated nabilone tablet has an in vitro dissolution profile such that:
about 97% of the pharmaceutically active ingredient is released after 15 min, and
about 98% of the pharmaceutically active ingredient is released after 30 min, as measured by USP Type II Apparatus, with 1000 ml of 0.1% tween 80 media at 37° C. that is resulting in vivo Cmax and AUC values between 80% to 125% of the an equivalent dose of a Cesamet® tablet .

In a further aspect of the present invention the orally disintegrated nabilone tablet, provides maximum plasma concentrations (Cmax) T/R ratio about 80% to about 125% and AUCt T/R ratio from about 82% to about 98% (with 90% confidence interval) in bioequivalence studies comparing to a reference product Cesamet®.

In an embodiment of the present invention the orally disintegrated nabilone tablet which the rate of absorption (Cmax) between 80% to 125% of that obtained with an equivalent dose of a Cesamet® tablet and the extent of absorption (AUCT) between 80% to 125% of that obtained with an equivalent dose of a Cesamet® tablet.

Another aspect of the present invention provides a method of manufacturing an orally disintegrating tablet of nabilone or a pharmaceutically acceptable salt thereof, comprises the following steps:
1. Preparation of granulation solution
   a) dissolving nabilone and povidone K30 in dehydrated alcohol and preparing granulating solution;
2. Granulation
   b) mannitol passing through comil;
   c) granulating the mixture with the solution;
3. Drying
   d) drying the wet granules and milling the dried granules;
   e) screening dried granules;
4. Extra -granular mixing
   f) adding dried granules to a bin blender;
   g) adding mannitol SD200, calcium silicate and crospovidone to a bin blender and mixing;
5. Lubrication
   h) adding magnesium stearate to this mixture and blending them;
6. Compression
   i) compressing the blended mixture to form tablets.

### DETAILED DESCRIPTION OF THE INVENTION

The present invention relates to a pharmaceutical composition comprising oral disintegrating tablet formulation containing nabilone or a pharmaceutically acceptable salt thereof, as an active ingredient using wet granulation method allows to obtain ODT nabilone dosage form that improved patient compliance, in particular with undergoing chemotherapy patients, including pediatric and geriatric patients.

The term “oral disintegrated tablet (ODT)”, as referred to herein, is defined to mean oral pharmaceutical compositions which when administered disintegrate/dissolve in the mouth rapidly without administering extra water and releases the active ingredient at very short period of time. By administering the orally disintegrating dosage forms, faster absorption of the drug occurs through buccal mucosa and it may reduce the first pass metabolism leading to better efficacy of the drug. These dosage forms provide the convenience of a tablet formulation while allowing the ease of swallowing. Such dosage forms due to their ease of administration and pleasant mouth feel, may encourage patients especially children, the elderly and patients who have difficulty in swallowing conventional tablets to adhere to daily medication regimens and also allow the luxury of much more accurate dosing. Yet another situation where such tablets would be useful is where water may not be readily available to assist in swallowing the tablet in specific conditions.

Because the tablets disintegrate inside the mouth, drugs may be absorbed in the buccal, pharyngeal, and gastric regions. Thus, rapid drug therapy intervention and increased bioavailability of drugs are possible. Because the pre-gastric drug absorption avoids the first-pass metabolism, the drug dose can be reduced if a significant amount of the drug is lost through the hepatic metabolism. ODTs are also called as Oro-disperse, mouth dissolving, rapidly disintegrating, fast melt, quick dissolve and freeze dried wafers

The term “active ingredient” and “active pharmaceutical ingredient” refers to an Active Pharmaceutical Ingredients (API) which are active chemicals used in the manufacturing of drugs. The active agent can be a therapeutic, a prophylactic, or a diagnostic agent.

Drug release and drug release profiles are measures or representations of the manner and timing by which a formulation releases or delivers active ingredients (drug) to a receiving environment (e.g. buccal mucosa, the stomach, intestines, etc.) upon administration. Various methods are known for evaluating drug release and producing release profiles, including in vitro tests which model the in vivo behavior of a formulation. These include USP dissolution testing for solid dosage forms.

Measures of bioavailability are well known in the art and include the area under the plasma concentration-time curve (AUC), the concentration maximum (Cₘₐₓ), and the time to Cₘₐₓ AUC is a measurement of the area under the plasma concentration-time curve, and is representative of the amount of drug absorbed following administration of a single dose of a drug (for example, see Remington: The Science and Practice of Pharmacy, (Alfonso R. Gennaro ed. 2000), page 999).

Cₘₐₓ is the maximum plasma concentration achieved after oral drug administration (Remington, page 999). An oral drug administration results in one Cₘₐₓ, but may result in greater than one “peak plasma concentration” or “plasma concentration peak” (for example, following the administration of a pulsed dose formulation).

Tₘₐₓ is the amount of time necessary to achieve the Cₘₐₓ after oral drug administration, and is related to the rate of absorption of a drag (Remington, page 999).

Bioequivalence is the absence of a significantly different rate and extent of absorption in the availability of the active ingredient when administered at the same dose under similar conditions. Bioequivalence can be measured by pharmacokinetic parameters such as, for example, AUC and Cₘₐₓ. Two compositions can be considered as “bioequivalent” if the 90% Confidence Interval of the relative mean Cmax and AUC of the test to reference is within 80.00% to 125.00%.

ODT's disintegration time target should be less than 30 seconds with good mouth feel and a friability that did not exceed 1%. To meet orally disintegrating tablets requirements, one could consider compressing tablets at lower hardness without comprising the friability of the tablets.

The main challenge for developing of orally disintegrating tablets is in the choice of excipient. Highly water soluble diluents can help improving the disintegration of tablets. Tablet compressed at lower hardness may have high friability on the other hand, high hardness may prolong disintegration time.

According to the present invention, the orally disintegrated nabilone tablet formulation is achieved by pharmaceutical composition containing:
(i) an intra-granular fraction comprising:
   a) at least one pharmaceutically active ingredient; and
   b) at least one pharmaceutically acceptable excipient, and
(ii) an extra-granular fraction comprising:
   a) at least one filer;
   b) at least one disintegrant, and
   c) at least one other pharmaceutically acceptable excipient.

According to the present invention, pharmaceutically active ingredient is nabilone or a pharmaceutically acceptable salt thereof that is present in an amount ranging from 0.01 mg to 10.0 mg. Preferably, nabilone is present in an amount of 0.1 mg to 5.0 mg, more preferred of 0.25 mg to 1.0 mg.

The pharmaceutical composition of the present invention, in addition to an active ingredient, contains pharmaceutically acceptable excipients added to the composition for a variety of purposes. At least one pharmaceutically acceptable excipient may be present in the composition of the present invention, such as for example diluents, binders, disintegrants, lubricants, glidants, sweeteners, and combination thereof. As understood by a person skilled in the art, these excipients are conventional excipients which are well known in the pharmaceutical art.

Suitable diluent or filler is selected from the group consisting of: mannitol, microcrystalline cellulose, lactose, starch, sodium carbonate, sodium bicarbonate, calcium carbonate, magnesium carbonate, sorbitol, xylitol and mixtures thereof.

Preferably, the filler is mannitol and the amount is ranges from about 10% to about 90% w/w of the pharmaceutical composition. More preferably, in the intra-granular fraction is ranges from about 10.0% to about 70.0% w/w and in the extra-granular fraction from about 10.0% to about 30.0% w/w of the pharmaceutical composition.

According to one embodiment of the invention, the orally disintegrating tablet formulation of nabilone comprises mannitol, wherein it is present in an amount of between 10.0% to 90.0% by weight, preferably it is 10.0% to 85.0% by weight of the total tablet weight.

Suitable disintegrant is selected from the group consisting of: microcrystalline cellulose, starches, sodium starch glycolate, croscarmelose sodium, crospovidone, calcium silicate, and a combination thereof.

According to the present invention, is established that crospovidone alone as disintegrant is not sufficient to get a good disintegration. Preferably, the disintegrant is a combination of crospovidone and calcium silicate in the extra-granular fraction. More preferable, the amount of the disintegrant in optimized proportion is ranges from about 2.0% to about 20.0% w/w of the pharmaceutical composition. More preferably, the weight ratio of crospovidone and calcium silicate is in the range of 1:2 and 2:1.

Suitable binder is selected from the group consisting of: hydroxypropyl cellulose, hydroxypropylmethyl cellulose, carboxymethyl cellulose, methyl cellulose, hydroxyethyl cellulose, carboxyethyl cellulose, carboxymethylhydroxyethyl cellulose, polyvinyl pyrrolidone, polyethylene glycol, polyvinyl alcohol, polymethacrylates, and a combination thereof.

Preferably, the binder is povidone K30 and the amount is ranges from about 1.0% to about 5.0% w/w of the pharmaceutical composition. More preferably, the intra-granular fraction contains from 2.0% to about 4.0% w/w of the pharmaceutical composition.

Suitable lubricant is selected from the group consisting of: magnesium stearate, calcium stearate, zinc stearate, sodium stearate, stearic acid, aluminum stearate, sodium stearyl fumerate, glyceryl behenate, hydrogenated vegetable oil and combinations thereof.

Preferably, the lubricant is magnesium stearate and is present in an amount ranging from about 0.1% w/w to about 3.0% w/w of the total composition.

According to the present invention, the orally disintegrated nabilone tablet formulation is achieved by pharmaceutical composition comprising:
(i) an intra-granular fraction comprising:
   a) from 0.1% to 0.5% w/w of nabilone or pharmaceutically acceptable salt thereof;
   b) from 5.0% to 70.0% w/w of spray-dried mannitol,
   (c) from 1.0% to 5.0% w/w of povidone K30, and
(ii) an extra-granular fraction comprising:
   d) from 5.0% to 30.0% w/w of spray-dried mannitol,
   e) from 1.0% to 20.0% w/w of crospovidone XL,
   f) from 1.0% to 10.0% w/w of calcium silicate, and
   g) from 0.1% to 2.0% w/w of magnesium stearate, wherein said orally disintegrated composition resulted in a stable, uniform and bioequivalent formulation compared to the reference product Cesamet®.

Oral dosage forms which may be employed with the present invention include granules, spheroids or pellets, tablets, a capsule or in any other suitable solid form. Preferably, however the oral dosage form is a tablet.

It is difficult to develop orally disintegrating compositions because of several different reasons. First of all, the time in which dosage form must disintegrate in the oral cavity with the existence of saliva has to be much shorter than it should be in stomach. So those compositions should be very porous and should not be very hard. These porous compositions tend to be very sensitive to humidity. As a consequence, they may have some stability problems. Additionally, orally disintegrating compositions need to take precautions in the preparation, packaging, handling and storing of the finished dosage forms.

The orally disintegrating pharmaceutical compositions of the present invention may be manufactured by conventional technology well known to those skilled in the art such as wet granulation, direct compression, dry granulation and the like. The orally disintegrating compositions of the present invention may also be manufactured by other technologies such as zydis, orasolv, durasolv, wowtab and the like.

Wet granulation technique results in cores of a high hardness which make it difficult to obtain fast dissolving and fast disintegrating tablets. Wet granulation leads to coarse dispersions in the oral cavity resulting in a poor patient compliance. The use of solvents and the additional drying step make this technique expensive.

Direct compression is a commonly used tablet manufacturing process to produce orally disintegrating tablets. Because it uses existing high-speed tablet press equipment and common excipients, it is often preferred over other manufacturing processes for orally disintegrating tablets. A direct-compression formulation has better physical properties relative to other methods that may eliminate the need for special packaging.

The direct compression formulation and solid oral dosage form of the present invention may further comprise other optional ingredients as desired, including natural and/or artificial sweeteners such as taste-masking agents and/or flavors such as menthol, and colorants (e.g., red iron oxide dye), and other processing aids may be employed as needed or desired to facilitate handling and/or compression into tablets or other oral dosage forms.

The manufacturing process according to the present invention comprises following steps:

Step 1: Preparation of granulation solution

Step 2: Granulation

Step 3: Drying

Step 4: Extra-granular mixing

Step 5: Lubrication

Step 6: Compression

The dosage forms of the present invention may facilitate enhanced absorption of the nabilone through oral mucosa and reduced ingestion of the cannabinoid. In the case of certain cannabinoids, this means that there may be considerably less oxidative degradation of the cannabinoid resulting in improved therapeutic effect with reduced psychotropic effect.

The present invention provides an orally disintegrating tablet comprising nabilone and at least one pharmaceutically acceptable excipient, wherein the total weight of nabilone is about 0.01% to 0.5% by weight of the total tablet and wherein the tablet disintegrates within up to 30 seconds and less than 60 seconds in oral cavity and does not exhibit a food effect when ingested by a patient that has eaten.

Stability data in ALU/ALU cold forming blister at 40° C. and 75% RH, shows that these oral disintegrating pharmaceutical compositions of nabilone exhibit good stability.

The following examples illustrate the preferred embodiments and various aspects of the present invention and are not be considered as limiting the invention in any way.

### EXAMPLE 1

### Nabilone Orally Disintegrating Tablet and Method of Manufacturing

The Manufacturing process comprises following steps:

Step 1: Preparation of a Granulation Solution

The required quantity of the nabilone and povidone K30 (see Table 1) are dissolved in dehydrated alcohol under stirring at room temperature. Stirring is continued until a clear solution is obtained.

Step 2: Granulation

Mannitol SD100 is passed through suitable comil equipped screen at slow speed then is added to high shear granulator in required quantity. The granulating solution of step 1 is added to the high shear bowl under mixing.

Step 3: Drying

The wet granules of step (2) are dried in a fluid bed until an LOD value less than 1% is obtained. Then, dried granules of previous step are screened through suitable screen to obtain uniform granules.

Step 4: Extra Granular Mixing

The screened granules of step (3) are added to a bin blender and blended with mannitol SD200, calcium silicate and crospovidone XL (see Table 1). These ingredients are dispersed in a bin blender for 1 min, passed through comil equipped suitable sieve then added to the blend of previous step and blended for 10 minutes.

Step 5: Lubrication

Magnesium stearate screened through suitable sieve and blended with blend of step (4).

Step 6: Compression

The obtained blend is compressed on a compression machine.

The formulation and manufacturing steps of Example 1 is set out in Table 1.

TABLE 1
Nabilone Formulation and Manufacturing steps.

| | | Example 1 | | |
|---|---|---|---|---|
| S. No. | Ingredient | Function | mg/unit | % w/w |

Intra-granular fraction

| | | | | |
|---|---|---|---|---|
| 1 | mannitol (spray dried) | filler | 170.0 | 56.7 |

Granulation

| | | | | |
|---|---|---|---|---|
| 2 | nabilone | API | 1.0 | 0.3 |
| 3 | povidone K30 | binder | 9.0 | 3.0 |
| 4 | dehydrated alcohol | granulating solvent | | |

Extra -granular fraction

| | | | | |
|---|---|---|---|---|
| 5 | mannitol (spray dried) | filler | 70.5 | 23.5 |
| 6 | calcium silicate | disintegrant | 15.0 | 5.0 |
| 7 | crospovidone XL | disintegrant | 30.0 | 10.0 |

Lubrication

| | | | | |
|---|---|---|---|---|
| 8 | magnesium stearate | lubricant | 4.5 | 1.5 |
| | Total | | 300.0 | 100 |

Content uniformity of tablets is evaluated for 10 individual tablets and the results are summarized in Table 2.

TABLE 2
Content uniformity results of nabilone tablets of Example 1.
Example 1

| sample | % LC |
|---|---|
| 1 | 97.9 |
| 2 | 99.0 |
| 3 | 99.9 |
| 4 | 98.1 |
| 5 | 99.1 |
| 6 | 97.2 |
| 7 | 98.5 |
| 8 | 97.5 |
| 9 | 98.9 |
| 10 | 99.0 |
| Average | 98.5 |
| Acceptance value (L1) | 2% (conforms) |

### Stability Information of an Orally Disintegrating Tablets of Nabilone

Tablets manufactured as per Example 1 further are tested to evaluate stability of packaged finished product. A comparative stability data is summarized in Table 3.

TABLE 3
COMPARATIVE STUDY ON STABILITY
Pack: Tablets packed in ALU/ALU blisters
Stability condition: 40° C. ± 2° C./75% ± 5% RH for 6 months

| | Specification | Initial analysis | |
|---|---|---|---|
| | limit | (T = 0) | 6 M/40° C./75% RH |
| Assay | 90-110% | 98.6 | 97.5 |
| Dissolution in | NLT80Q in | 98.0 | 95.0 |
| 1000 ml 0.1% | 30 min | | |
| tween 80 | | | |

Known degradation product

| | | | |
|---|---|---|---|
| Compound 1 | NMT 0.8% | <0.06 | <0.06 |
| Individual | NMT 0.3% | <0.06 | 0.12% RRT 0.44 |
| unspecified | | | 0.14% RRT 0.95 |
| products | | | 0.08% RRT 1.38 |
| | | | 0.09% RRT 1.48 |

Note:
For tested formulation degradation products (known and unknown) are below the reporting thresholds during Initial analysis.

Compound 1: 5-(1′1-dimethylheptyl)-resorcinol

RRT: relative retention time

### Evaluation of Dissolution Profile

The orally disintegrated tablets of nabilone obtained from Example 1 are subsequently tested for in vitro dissolution rate, measured by Apparatus (USP Type II), using the following parameters:

Media: 0.1% tween 80

Volume: 1000 ml

Temperature: at 37 deg.C

The dissolution results are set out in Table 4.

TABLE 4
Dissolution rate of nabilone orally disintegrating tablet of
Example 1 to the reference product Cesamet ®.

| Time | Example 1 (1 mg) | Cesamet ® (1 mg) |
|---|---|---|
| (min) | Commutative % released | Commutative % released |
| 10 | 97.0 | 87.0 |
| 15 | 97.0 | 94.0 |
| 30 | 98.0 | 97.0 |
| 45 | 98.0 | 98.0 |

### Comparative Bioequivalence Study

The pharmaceutical composition obtained from above mentioned Example 1 was subsequently tested in a bioequivalence study. A pilot bioequivalence study was conducted in 10 healthy volunteers, in a single center. The orally disintegrating tablets of nabilone (1 mg tablet) of the present invention are compared to Cesamet® (1 mg capsule) in fast conditions. The bioequivalence study data, single dose, randomized, blinded, 2 periods, 2 sequences, cross over design shows results in Table 5.

TABLE 5
Bioequivalence study data of Nabilone for Example 1.

| | Intra- | | | | 90% confidence | |
|---|---|---|---|---|---|---|
| | subject | Geometric LS means | | | limits | |
| Parameter | CV (%) | Test | Reference | Ratio | Lower | Upper |
| Cₘₐₓ | 26.45 | 1561.91 | 1566.47 | 99.71 | 80.32 | 123.78 |
| AUC_{T} | 10.91 | 2259.52 | 2514.33 | 89.87 | 82.09 | 98.38 |
| AUCᵥ | 10.73 | 2372.19 | 2620.43 | 90.53 | 82.82 | 98.95 |

Conclusion: the test product, orally disintegrated formulation of nabilone, when compared to the reference product Cesamet ®, met the bioequivalence criteria with respect to rate of absorption (Cmax) and the extent of absorption (AUCt).

### EXAMPLE 2

### Disintegration Time Optimization for ODT

Manufacturing Process comprising following steps:

Step 1: The required quantity of the pregelatinized starch is passed through suitable sieve and introduced into high shear bowl. The blend is mixed for 5 minutes.

Step 2: The required quantity of the polyvinyl pyrrolidone K30 is dissolved in appropriate amount of dehydrated alcohol under stirring conditions. The clear solution obtained is used to granulate step (1) blend.

Step 3: The wet granules of step (2) are dried in a fluid bed dryer then passed through comill fitted with appropriate sieve.

Step 4: The required quantity of the mannitol and crospovidone XL10 are dispersed manually in a polyethylene bag for 1 minute and passed through suitable mesh sieve.

Step 5: The screened granules of step (3) and blend of step (4) are mixed in a bin blender for 10 minutes.

Step 6: The required quantity of the magnesium stearate is sifted through suitable mesh sieve and dispersed with certain amount of step (5) blend. The dispersion is then added to the balance of step (5) blend and mixed for 3 minutes.

Step 7: The obtained final blend is compressed in a rotary press using round flat face bevel edge punches to get 400 mg tablet weight.

The formulation and manufacturing steps of Example 2 (placebo) are set out in Table 6.

TABLE 6
Formulation and Manufacturing steps for Example 2 (placebo).

| | | | |
|---|---|---|---|
| No | Name of excipient | Mg/tab | % w/w |

Intra-granular fraction

| | | | |
|---|---|---|---|
| 1 | polyvinyl pyrrolidone K30 | 9.0 | 2.3 |
| 2 | pregelatinized starch | 200.0 | 50.0 |
| 3 | dehydrated alcohol | q.s | |

Extra-granular fraction

| | | | |
|---|---|---|---|
| 4 | mannitol | 126.0 | 31.5 |
| 5 | crospovidone XL10 | 60.0 | 15.0 |
| 6 | magnesium stearate | 4.0 | 1.0 |
| | Tablet weight | 400.0 | 100 |

Observation: The disintegration time of tablets from Example 2 is more than 2 minutes.

Conclusion: The tablet formulation from Example 2 did not meet the disintegration time criteria for orally disintegrating tablets which is less than 30 seconds.

### EXAMPLE 3

### Disintegration Time Optimization for ODT Formulation

For Example 3 formulation, tablet's weight is reduced from 400 mg to 210 mg and amount of pregelatinized starch is reduced in the formulation.

The formulation and manufacturing steps of Example 3 is set out in Table 7.

TABLE 7
Formulation and Manufacturing steps for Example 3 (placebo).

| | | | |
|---|---|---|---|
| No | Name of excipient | Mg/tab | % w/w |

Intra-granular fraction

| | | | |
|---|---|---|---|
| 1 | pregelatinized starch | 80.0 | 38.1 |
| 2 | polyvinyl pyrrolidone K30 | 9.0 | 4.3 |
| 3 | dehydrated alcohol | q.s | |

Extra-granular fraction

| | | | |
|---|---|---|---|
| 4 | mannitol | 97.9 | 46.6 |
| 5 | crospovidone XL | 20.0 | 9.5 |
| 6 | magnesium stearate | 2.1 | 1.0 |
| | Tablet weight | 210.0 | 100 |

The manufacturing process is the same as per Example 2.

Observation: For Example 3 formulation, disintegration time is 1.30 seconds.

Conclusion: The tablets from Example 3 did not meet the disintegration time criteria for orally disintegrating.

### EXAMPLE 4

### Disintegration Time Optimization for Nabilone ODT

For Example 4 formulation tablet's weight is reduced from 400 mg to 320 mg. The mannitol is present in intra-granular fraction and in extra-granular fraction; also the crospovidone XL is added to intra-granular portion.

The formulation and manufacturing steps of Example 4 is set out in Table 8.

TABLE 8
Nabilone Formulation and Manufacturing steps for Example 4.

| | | | |
|---|---|---|---|
| No | Name of excipient | Mg/tab | % w/w |

Intra-granular fraction

| | | | |
|---|---|---|---|
| 1 | mannitol | 102.0 | 31.9 |
| 2 | pregelatinized starch | 80.0 | 25.0 |
| 3 | crospovidone XL | 16.0 | 5.0 |
| 3 | polyvinyl pyrrolidone K30 | 9.0 | 2.8 |
| 4 | Nabilone | 1.0 | 0.3 |
| 5 | Dehydrated Alcohol | q.s | |

Extra-granular fraction

| | | | |
|---|---|---|---|
| 6 | mannitol | 76.8 | 24.0 |
| 7 | crospovidone XL | 32.0 | 10.0 |
| 8 | magnesium stearate | 3.2 | 1.0 |
| | Tablet weight | 320.0 | 100 |

Manufacturing Process comprising following steps:

Step 1: The required quantity of the mannitol and pregelatinized starch (as indicate in Table 8) are added into high shear bowl and mix for 5 minutes.

Step 2: The required quantity of the polyvinyl pyrrolidone K30 is dissolved in appropriate amount of dehydrated alcohol under stirring conditions. The clear solution obtained is used to granulate step (1) blend.

Step 3: The granules of step (2) are dried in a fluid bed dryer then passed through comill fitted with appropriate sieve.

Step 4: The required quantity of the mannitol and crospovidone XL are dispersed manually in a polyethylene bag for 1 minute and passed through suitable mesh sieve.

Step 5: The screened granules of step (3) and blend of step (4) are mixed in a bin blender for 10 minutes.

Step 6: The magnesium stearate is sifted through suitable mesh sieve and dispersed with an amount of blend of step (5). The dispersion is then added to the balance of step (5) blend and mixed for 3 minutes.

Step 7: The obtained final blend is compressed in a rotary press using round flat bevel edged punches.

Observation: The disintegration time of nabilone tablets from Example 4 is more than 1 minute.

Conclusion: The tablets from Example 4 did not meet the disintegration time criteria for orally disintegrating.

### EXAMPLE 5

### Nabilone ODT-Disintegration Time Optimization

The pregelatinized starch was removed from the formulation of Example 5. Mannitol is present in intra-granular fraction and in extra-granular fraction; also the crospovidone XL is added to intra-granular fraction.

The formulation and manufacturing steps of Example 5 is set out in Table 9.

TABLE 9
Nabilone Formulation and Manufacturing steps for Example 5.

| | | | |
|---|---|---|---|
| No | Name of excipient | Mg/tab | % w/w |

Intra-granular fraction

| | | | |
|---|---|---|---|
| 1 | nabilone | 1.0 | 0.3 |
| 2 | mannitol | 182.0 | 56.9 |
| 3 | crospovidone XL | 16.0 | 5.0 |
| 4 | polyvinyl pyrrolidone K30 | 9.0 | 2.8 |
| 5 | dehydrated alcohol | q.s | |

Extra-granular fraction

| | | | |
|---|---|---|---|
| 6 | mannitol | 76.8 | 24.0 |
| 7 | crospovidone XL | 32 | 10.0 |
| 8 | magnesium stearate | 3.2 | 1.0 |
| | Tablet weight | 320.0 | 100 |

The manufacturing process for Example 5 is same as for example 4. The granulating solution is made by dissolving Nabilone and polyvinyl pyrrolidone in dehydrated alcohol under stirring.

Observation: For Example 5 formulation, disintegration time is between 42 seconds to 1 minute.

Conclusion: The tablets from Example 5 did not meet the disintegration time criteria for orally disintegrating.

### EXAMPLE 6

### Nabilone ODT Formulation

The pregelatinized starch was removed from the formulation of Example 6 and tablet weight increased to 400 mg. The mannitol is present in intra-granular fraction and in extra-granular fraction.

The formulation and manufacturing steps of Example 6 is set out in Table 10.

TABLE 10
Nabilone Formulation and Manufacturing steps for Example 6.

| | | | |
|---|---|---|---|
| No | Name of excipient | Mg/tab | % w/w |

Intra-granular fraction

| | | | |
|---|---|---|---|
| 1 | Mannitol | 250.0 | 62.5 |
| 2 | Polyvinyl pyrrolidone K30 | 9.0 | 2.25 |
| 3 | Nabilone | 1.0 | 0.25 |
| 4 | Dehydrated Alcohol | q.s | |

Extra-granular fraction

| | | | |
|---|---|---|---|
| 5 | Mannitol | 74.0 | 18.5 |
| 6 | Calcium Silicate | 20.0 | 5.0 |
| 7 | Crospovidone | 40.0 | 10.0 |
| 8 | Magnesium Stearate | 6.0 | 1.5 |
| | Tablet weight | 400.0 | 100 |

### Bioequivalence Results For Example 6—Nabilone ODT

TABLE 11
Comparison of Results with Standard for Bioequivalence Nabilone

| | | GEOMETRIC | | | | |
|---|---|---|---|---|---|---|
| | | LS MEANS | | | 90% CONFI- | |
| PARAM- | Intra- | | REFER- | | DENCE LIMITS | |
| ETER | CV, % | TEST | ENCE | RATIO | LOWER | UPPER |
| Cₘₐₓ | 29.5 | 1601.0 | 1345.5 | 118.99 | 106.54 | 132.90 |
| AUC_{0-T} | 15.0 | 2623.2 | 2640.1 | 99.36 | 93.85 | 105.19 |
| AUC_{0-∞} | 15.9 | 2718.7 | 2785.2 | 97.61 | 91.73 | 103.87 |

TABLE 12
Comparison of Nabilone formulations for ODT

| | | | | | | EXTRA-GRANULAR % w/w | | | | |
|---|---|---|---|---|---|---|---|---|---|---|
| No of | INTRA-GRANULAR % w/w | | | | | | | Mg | Ca | Disint. |
| Example | Nabilone | Mannitol | Povidone | Starch | Crospovidone | Mannitol | Crospovidone | stear. | silicate | time |
| Ex. 1 | 0.3 | 56.7 | 3.0 | 0 | 0 | 23.5 | 10.0 | 1.5 | 5.0 | <30″ |
| Ex. 2 | 0 | 0 | 2.3 | 50.0 | 0 | 31.5 | 15.0 | 1.0 | 0.0 | >2′ |
| Ex. 3 | 0 | 0 | 4.3 | 38.1 | 0 | 46.6 | 9.5 | 1.0 | 0.0 | >1′ |
| Ex. 4 | 0.3 | 31.9 | 2.8 | 25.0 | 5.0 | 24.0 | 10.0 | 1.0 | 0.0 | >1′ |
| Ex. 5 | 0.3 | 56.9 | 2.8 | 0 | 5.0 | 19.2 | 8.0 | 0.8 | 0.0 | 42″ |
| | | | | | | | | | | to 1′ |
| Ex. 6 | 0.3 | 62.5 | 2.2 | 0 | 0 | 18.5 | 10.0 | 1.5 | 5.0 | <30″ |

Conclusion: The tablets from Example 1 and 6 meet the disintegration time criteria for orally disintegrating formulation.

1. An orally disintegrating pharmaceutical composition comprising nabilone or a pharmaceutically acceptable analog, derivative, prodrug or salt thereof, wherein the composition disintegrates in the mouth of a patient within 30 seconds of administration.

2. The orally disintegrating pharmaceutical composition according to claim 1 comprising:
(i) an intra-granular fraction comprising:
a) nabilone or a pharmaceutically acceptable salt thereof, and
b) at least one pharmaceutically acceptable excipient;
and (ii) an extra-granular fraction comprising:
c) mannitol;
d) at least one disintegrant, and
e) at least one other pharmaceutically acceptable excipient,
wherein the orally disintegrating pharmaceutical composition disintegrates within up to 30 seconds as per USP.

3. The orally disintegrating pharmaceutical composition according to claim 1, wherein nabilone or a pharmaceutically acceptable salt thereof is present in an amount ranging from 0.25 mg to 5.0 mg or 0.25 mg to 2.0 mg.

4. The orally disintegrating pharmaceutical composition according to claim 1, wherein nabilone or a pharmaceutically acceptable salt thereof is present in an amount from 0.25 mg to 0.5 mg or 0.25 mg to 1.0 mg.

5-6. (canceled)

7. The orally disintegrating pharmaceutical composition according to claim 1, wherein the composition comprises nabilone along with at least one pharmaceutically acceptable excipient selected from the group consisting of: binders, fillers, diluents, disintegrants, taste masking agents, sweeteners, lubricants, stabilizers, coating polymers and combinations thereof

8. The orally disintegrating pharmaceutical composition according to claim 1, wherein the composition further comprises a mannitol present in amount ranging from 10.0% to 90.0% w/w of the total composition.

9. The orally disintegrating pharmaceutical composition according to claim 8, wherein the mannitol is present in the intra-granular fraction in an amount ranging from 10.0% to 70.0% w/w of the total composition.

10. The orally disintegrating pharmaceutical composition according to claim 8, wherein the mannitol is present in the extra-granular fraction in an amount ranging from 10.0% to 30.0% w/w of the total composition.

11. The orally disintegrating pharmaceutical composition according to claim 7, wherein the composition comprises a binder selected from the group consisting of: hydroxypropyl cellulose, hydroxypropylmethyl cellulose, carboxymethyl cellulose, methyl cellulose, hydroxyethyl cellulose, carboxyethyl cellulose, carboxymethylhydroxyethyl cellulose, polyvinyl pyrrolidone, polyethylene glycol, polyvinyl alcohol, polymethacrylates and a combination thereof.

12. The orally disintegrating pharmaceutical composition according to claim 11, wherein the binder is povidone K30 and is present in the intra-granular fraction in an amount ranging from 1.0% to 5.0% w/w of the total pharmaceutical composition.

13. The orally disintegrating pharmaceutical composition according to claim 7, wherein the composition comprises a disintegrant selected from the group consisting of: microcrystalline cellulose, starches, sodium starch glycolate, croscarmelose sodium, crospovidone, povidone, calcium silicate and a combination thereof, which is present in an amount ranging from 2.0% to 20.0% w/w of the pharmaceutical composition.

14. The orally disintegrating pharmaceutical composition according to claim 2, wherein the extra-granular fraction comprises crospovidone in an amount ranging from 2.0% to 20.0% w/w of the total pharmaceutical composition.

15. The orally disintegrating pharmaceutical composition according to claim 14, wherein the extra-granular fraction further comprises calcium silicate in an amount ranging from 2.0% to 15.0% w/w of the total pharmaceutical composition.

16. The orally disintegrating tablet pharmaceutical composition according to claim 2, wherein the at least one disintegrant in the extra-granular fraction is in the weight ratio ranging of 1:2 and 2:1.

17. The orally disintegrating pharmaceutical composition of claim 1 comprising:
(i) an intra-granular fraction comprising:
a) from 0.1% to 0.5% w/w of nabilone or pharmaceutically acceptable salt thereof;
b) from 5.0% to 70.0% w/w of spray-dried mannitol,
(c) from 1.0% to 5.0% w/w of povidone K30, and
(ii) an extra-granular fraction comprising:
d) from 5.0% to 30.0% w/w of spray-dried mannitol,
e) from 1.0% to 20.0% w/w of crospovidone,
f) from 1.0% to 10.0% w/w of calcium silicate, and
g) from 0.1 to 2.0% w/w of magnesium stearate,
wherein said orally disintegrated pharmaceutical composition has an in vitro dissolution profile by which more than 90% of the active ingredient is released after 10 minutes, as measured with a USP Type II apparatus with 1000 ml of 0.1% tween 80 buffer at 37° C.

18. The orally disintegrating pharmaceutical composition according to claim 17, wherein the in vitro dissolution profile of the composition provides more than 95% of the active ingredient released after 15 minutes, as measured by USP.

19. The orally disintegrating pharmaceutical composition according to claim 17, wherein the composition is in the form of a tablet, a capsule, granules, pellets, caplets, mini-tablets, a capsule filled with mini-tablets and/or pellets, a multi-layer tablet, granules for suspension, or granules powder filled in a sachet.

20. The orally disintegrating pharmaceutical composition according to claim 19, wherein the tablet exhibits oral disintegratability less than 30 seconds.

21-22. (canceled)

23. A process for the manufacturing of an orally disintegrating pharmaceutical composition comprising nabilone, or a pharmaceutically acceptable salt thereof according to claim 2, comprising the steps of:
a) dissolving nabilone and povidone K30 in a dehydrated alcohol and preparing a granulating solution;
b) granulating the intra-granular fraction by mixing mannitol with the granulation solution from step (a);
c) drying the wet granules from step (b) and then screening the dried granules;
d) mixing the extra-granular fraction by adding the dried granules from step (c), mannitol SD200, calcium silicate and crospovidone XL to a bin blender and mixing;
e) blending the granules with magnesium stearate, and
f) compressing the blended mixture from step (e) to form tablet;
wherein, optionally, the orally disintegrable tablet comprises 0.1% to 0.5% w/w of nabilone or pharmaceutically acceptable salt thereof, 5.0% to 70.0% w/w of spray-dried mannitol, and of 1.0% to 5.0% w/w of povidone K30 in the intra-granular fraction, and of 5.0% to 30.0% w/w of spray-dried mannitol, of 1.0% to 20.0% w/w of crospovidone XL, and of 1.0% to 10.0% w/w of calcium silicate in the extra-granular fraction, and further a lubricant, wherein the orally disintegrating tablet exhibits stability requirements and disintegrate in oral cavity within less than 30 seconds.

24. (canceled)

25. A method of treating nausea and vomiting symptoms associated with chemotherapy in patients who have difficulty i swallowing conventional tablets, the method comprising administering an orally disintegrating pharmaceutical composition of claim 1.
